(19) United States
(12) Patent Application Publication
(10) Pub. No.: US 2012/0143033 A1
(43) Pub. Date: Jun. 7, 2012

(54) CENTER RIDGE ECG MONITORING ELECTRODE

(76) Inventors: Mark D. Tiegs, West Allis, WI (US); Edward J. Arndt, Brookfield, WI (US)

(21) Appl. No.: 12/958,131

(22) Filed: Dec. 1, 2010

(51) Int. Cl. A61B 5/0408

(52) U.S. Cl. 600/391

(57) ABSTRACT

An ECG electrode assembly is provided. The electrode assembly includes a backing element, an electrode element, and an electrolyte gel. The electrode assembly includes an electrode assembly having a connection portion or ridge located generally in the center of the backing element. The electrode assembly extends through an aperture in the backing element and includes an integrally formed sensing potion located on the side of the backing element opposite the connection portion.

### BACKGROUND OF THE INVENTION

Disposable electrodes applied to the skin of a patient are used to monitor the electrical activity of bodily functions. Electrocardiogram or ECG electrodes are positioned on a patient's body to gather biopotential electrical signals generated by the heart. These signals are generally transmitted to a monitor which produces a visual representation of the patient's heart condition.

The strength and accuracy of these signals is dependent on motion artifacts caused by the movement of the patient's skin relative to the electrode and/or electrode movement relative to the patient's skin. Such movement can cause extraneous signals, shifting the desired baseline.

Existing ECG monitoring electrodes fall into two categories based on the type of attachment mechanism utilized. The first category is tab electrodes in which the lead wires of the monitor are connected to the electrode through a tab element which extends generally from the perimeter of the electrode. The lead wires of the monitor are generally attached to the tab element by an alligator clip. Tab-type electrodes are typically used for short-term or resting monitoring uses. Tab-type electrodes are relatively inexpensive, however, the electrodes often move relative to the patient's skin due to forces applied to the electrode by the pulling on the lead wires. Such force is applied in a direction that is parallel to the surface of the skin and can cause the electrode to be moved relative to the skin or pulled off of the skin completely.

The second category is snap electrodes in which the lead wires of the monitor are connected to the electrode through a snap element which is generally located in the center of the electrode. The lead wires may either be snapped onto the snap element through a mating connection device or through an alligator clip clipped on to the snap element. A snap electrode typically utilizes a male connection on the snap element and a female connection on the lead wires. The generally center attachment point for the snap-type electrode is generally more stable than the tab-type electrode because any force applied to the electrode by pulling on the lead wire is applied in a direction that is perpendicular to the surface of the skin. This perpendicular force leads to less movement of the electrode relative to the skin than in tab-type electrodes, and therefore more accurate monitoring. However, such electrodes are relatively costly compared to the tab-type electrodes. Further, because the snap element protrudes outwardly from the base of the electrode, the snap-type electrodes take up more space, making shipping and storage more costly and inconvenient. Further, snap-type electrodes can cause discomfort to the patient due to the force required to snap the lead wire connection device on to the electrode.

### SUMMARY OF THE INVENTION

The present invention provides methods and apparatus for providing and using an electrode assembly having a center ridge connection element.

In one embodiment, the apparatus of the present invention includes a backing element having a first surface and an opposed second surface. The backing element has a first aperture extending through it from the first surface to the second surface. The first aperture is located generally in the center of the backing element and can be in any orientation. The apparatus also includes an electrode element having a connection portion and at least one sensing portion. The electrode element extends through the first aperture such that the connection portion is adjacent to the first surface and the sensing portion is adjacent to the second surface. The electrode element is a flexible substrate with a conductive coating on at least a portion. The apparatus includes a layer of electrolyte gel covering at least a portion of the second surface of the backing element. The apparatus includes a liner element removably attached to the second surface of the backing element. The layer of electrolyte gel is located between the second surface of the backing element and the liner element.

The apparatus may have a backing element made of cloth.

The apparatus may have an electrode element sensing portion extending away from the aperture in a first direction which is generally parallel to the second surface of the backing element.

The apparatus may have a flexible substrate made of vinyl.

The apparatus may have a conductive coating made of silver/silver chloride.

The apparatus may have a flexible substrate further made of a single piece of vinyl.

The apparatus may have a connection portion of the electrode element made of two layers of flexible substrate. The two layers of flexible substrate may be fused together.

The electrolyte gel of the apparatus may take the form of solid adhesive hydrogel.

The electrolyte gel of the apparatus may cover the electrode element sensing portion.

The electrolyte gel of the apparatus may cover substantially the entire second surface of the backing element.

The apparatus may have a backing element made of foam.

The apparatus may have a backing element made of vinyl.

The apparatus may have a backing element made of tape.

The electrode element of the apparatus may include a first sensing portion extending away from the aperture in a first direction generally parallel to the second surface of the backing element and a second sensing portion extending away from the aperture in a second direction generally parallel to the second surface of the backing element.

The electrolyte gel of the apparatus may cover substantially the entire first and second electrode element sensing portion.

The apparatus may include the portion of the backing element second surface that is not covered by electrolyte gel being covered by adhesive.

The electrolyte gel of the apparatus may take the form of liquid gel.

The apparatus may include an absorptive member coupled to the electrode element sensing portion, the absorptive member being sized and configured to cover substantially the entire electrode element sensing portions. A predetermined volume of liquid electrolyte gel may be provided in the absorptive member. The apparatus may further include the portion of the backing element second surface that is not covered by electrolyte gel being covered by adhesive.

### BRIEF DESCRIPTION OF THE DRAWINGS

FIG. 1 is a perspective view of an embodiment of an electrode assembly according to the present invention.

FIG. 2 is a top view of the electrode assembly of FIG. 1.

FIG. 3 is a cross sectional view of the electrode assembly of FIG. 1.

FIG. 4 is a perspective view of an embodiment of an alternate embodiment an electrode assembly according to the present invention.

FIG. 5 is a top view of the electrode assembly of FIG. 4.

FIG. 6 is a cross sectional view of the electrode assembly of FIG. 4.

FIG. 7 is a perspective view of an additional alternate embodiment of an electrode assembly according to the present invention.

FIG. 8 is a top view of multiple electrode assembly of FIG. 1 on a single liner.

FIG. 9 is a cross sectional view of an alternate embodiment of an electrode assembly, similar to the embodiment of FIG. 3, but utilizing an alternate electrode element.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

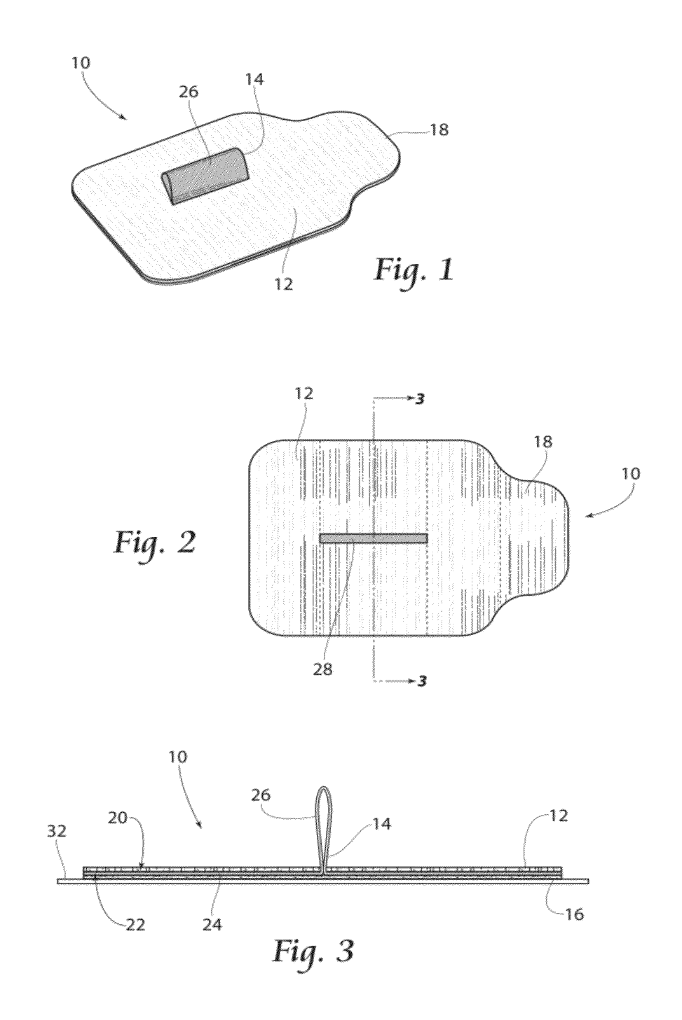

FIGS. 1 through 3 show an embodiment of an electrode assembly 10 according to the present invention. The electrode assembly 10 preferably includes a backing element 12, an electrode element 14 and a layer of electrolyte gel 16.

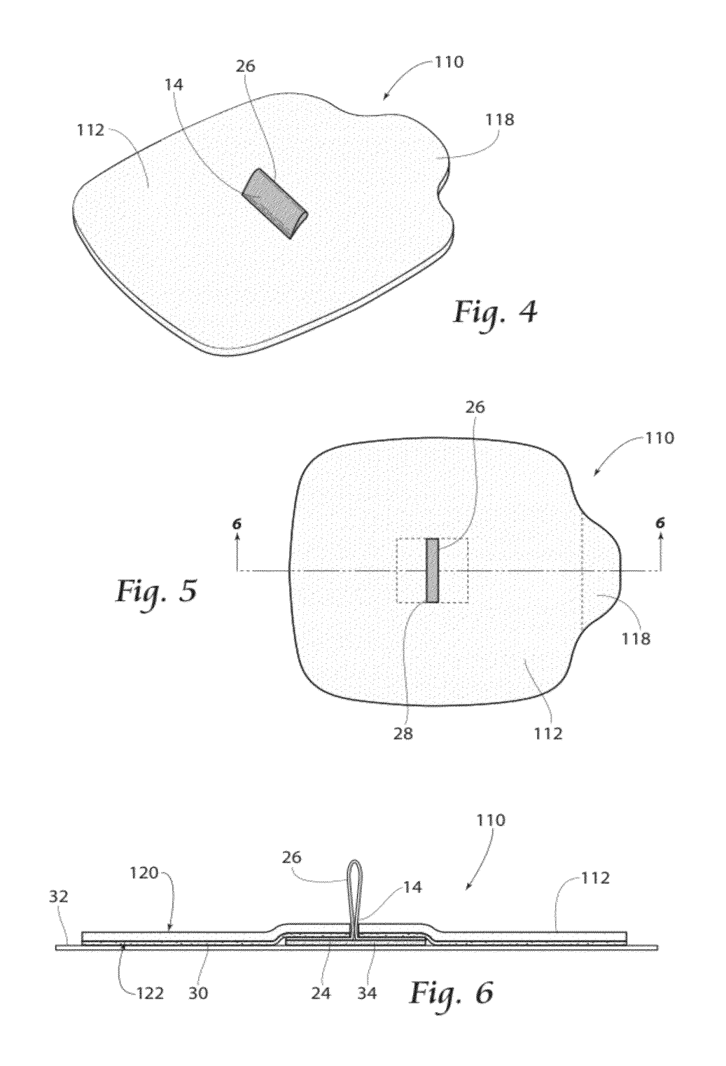

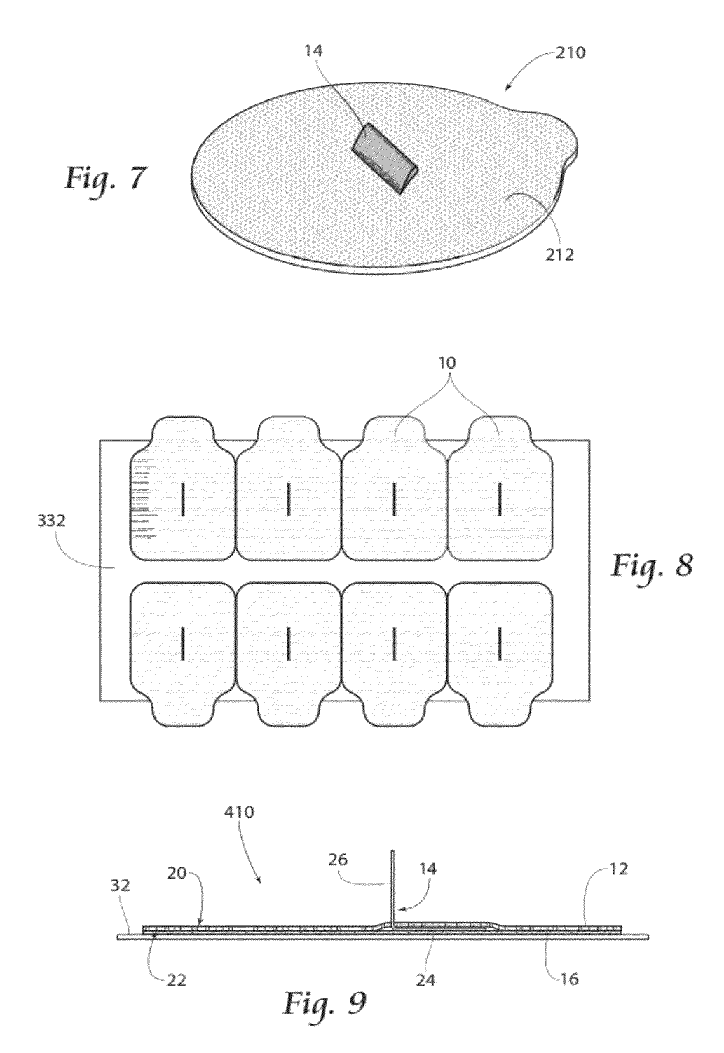

The backing element 12 may take any desired size and shape. In first the and second illustrated embodiments, the backing element 12,112 is generally rectangular; however it is also contemplated that the backing element 212 could be generally circular as shown in FIG. 7 or oval (not shown). The backing element 12,112 preferably includes a pull tab portion 18. The backing element 12,112 has a first surface 20 and an opposed second surface 22. The backing element 12,112 may be made of any material known in the art. In the illustrated embodiments the backing element 12,112 is preferably made of foam (FIGS. 4-6) or cloth (FIGS. 1-3).

Referring to FIG. 3, the electrode element 14 includes at least one sensing portion 24 and a connection portion 26. The backing element 12 preferably includes an aperture 28 extending therethrough to receive at least a portion of the electrode element 14. Preferably, the aperture 28 is generally at or near the center of the backing element 12. In the illustrated embodiment the aperture 28 is a slit. Preferably the electrode element 14 extends through the aperture 28 in the backing element 12 such that the connection portion 26 is adjacent the first surface 20 and the sensing portion 24 is adjacent the second surface 22. Preferably, the sensing portion 24 extends parallel to or along at least a portion of the second surface 22.

The electrode element 14 is preferably made of a substrate, at least a portion of which is coated with a conductive material. In the illustrated embodiment the substrate material is vinyl and the conductive material is a silver/silver-chloride coating, however it is contemplated that any substrate material and conductive material be any materials known in the art.

In the illustrated embodiment, the electrode element 14 is a single piece of substrate folded such that the connection portion 26 comprises a double layer of the substrate. Further the illustrated embodiment includes two sensing portions 24, each of which extends outwardly from the aperture 28 in the backing element 12 in opposite directions and extend along or parallel to the second surface 22 of the backing element 12. It is contemplated that, if desired, the two layers of the connection portion 26 may be fused together by any means known in the art including, but not limited to adhesive or heat fusing. It is contemplated that, if desired, the sensing portion 24 could extend in a single direction as shown in FIG. 9. Additionally, as also shown in FIG. 9, it is contemplated that the connection portion 26 could comprise a single layer of the substrate.

In the illustrated embodiment of FIGS. 1-4, the sensing portion 24 extends across the entire width of the second surface 22 of the backing element 12. However, it is contemplated that, if desired, the at least one sensing portion 24 may extend across only a portion of the second surface 22 of the backing element 112 as shown in the embodiment of FIGS. 4-6.

The connection portion 26 is sized and configured to make an electrical connection with the lead wires of a monitoring apparatus (not shown). It is contemplated that the lead wires of a monitoring apparatus may be coupled to the connection portion 26 by an alligator clip with attached lead wires (not shown). Although the connection portion 26 of the illustrated embodiment is generally rectangular, it is contemplated that the connection portion 26 may take any desired configuration.

An electrolyte gel 16 is preferably applied to at least a portion of the second surface 22 of the backing element 12. In the illustrated embodiment of FIGS. 1-3, the electrolyte gel 16 takes the form of a solid hydrogel. In the illustrated embodiment of FIGS. 1-3 the electrolyte gel 16 covers substantially the entire second surface 22 of the backing element 12. It should be understood that the sensing portion 24 of the electrode element 14 will be located between the second surface 22 and the electrolyte gel 16, and thus will be covered with the electrolyte gel 16. However it is contemplated that the electrolyte gel 16 may be present in only the area near the sensing portion 24 of the electrode element 14 as shown in FIGS. 4-7. In the embodiment of FIG. 1, the electrolyte gel 16 is an adhesive hydrogel. However it is contemplated that if the electrolyte gel 16 does not cover substantially the entire second surface 22 of the backing element 112 that an adhesive 30 could be applied to at least a portion of the remaining second surface 22 of the backing element 112 to allow the electrode to be adhered to a patient, as shown in FIGS. 4-7.

Preferably the electrode assembly 10 includes a liner element 32 covering the second surface 22, such that electrolyte gel is located between the liner element and the second surface 22. In the illustrated embodiment, the liner element 32 is sized and configured to cover substantially the entire second surface 22 of a single electrode assembly backing element 12. However, it is contemplated that the liner element 32 may comprise a single sheet of liner element 332 with multiple backing elements 12 provided thereon as shown in FIG. 8.

An alternative embodiment of an electrode assembly 110 according to the present invention is shown in FIGS. 4-6. The embodiment of FIGS. 4-6 is similar to the previously described embodiment; however in the embodiment of FIGS. 4-6 the electrolytic gel 16 is located only near the sensing element 14. The electrolytic gel 16 may be a solid hydrogel as described above, or it may be a liquid gel. If a liquid gel is utilized, the liquid gel is preferably applied to a carrying material, such as a sponge 34, which is attached to the sensing portion 24 of the electrode element 14. The sponge 34 may be secured to the sensing portion 24 of the electrode element 14 using any means known in the art including, but not limited to through adhesive.

Preferably, the sponge 34 is slightly larger than the sensing portion 24 of the electrode element 14. Preferably the second surface 22 of the pull tab 18 portion of the backing element is substantially free from adhesive 30 or electrolyte gel 16 to aid in applying and removing the electrode assembly 10,110,410 from a patient's skin. Alternatively, the pull tab 18 may have a backing member attached to the second surface there of 22.

While the illustrated embodiments show the aperture 28 and the connection portion 26 being configured laterally across the width of the electrode assembly 10 (FIGS. 1-3) or longitudinally along the length of the electrode assembly 110 (FIGS. 4-6), it should be understood that aperture 28 and connection portion 26 can take any orientation desired.

It is contemplated that the backing element may be made of any material known in the art including, but not limited to, foam, fabric, vinyl or tape.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. An apparatus comprising:
a backing element, the backing element having a first surface and an opposed second surface, the backing element having a first aperture therethrough, the first aperture extending from the first surface to the second surface, the first aperture being located generally in the center of the backing element and having any orientation;
an electrode element, the electrode element having a connection portion and at least one sensing portion, the electrode element extending through the first aperture such that the connection portion is adjacent to the first surface and the at least one sensing portion is adjacent to the second surface, wherein the electrode element is a flexible substrate with a conductive coating on at least a portion thereof;
a layer of electrolyte gel, the layer of electrolyte gel covering at least a portion of the backing element second surface: and
a liner element removably coupled to the backing element second surface, wherein the layer of electrolyte gel is located between the backing element second surface and the liner element.

2. The apparatus of claim 1 wherein the backing element is made of cloth.

3. The apparatus of claim 1 wherein the at least one electrode element sensing portion extends away from the aperture in a first direction generally parallel to the backing element second surface.

4. The apparatus of claim 1 wherein the flexible substrate is vinyl.

5. The apparatus of claim 1 wherein the conductive coating is silver/silver chloride.

6. The apparatus of claim 1 wherein the flexible substrate further comprises a single piece of vinyl.

7. The apparatus of claim 1 wherein the connection portion of the electrode element further comprises two layers of flexible substrate.

8. The apparatus of claim 7 wherein the two layers of flexible substrate are fused together.

9. The apparatus of claim 1 wherein the electrolyte gel is a solid adhesive hydrogel.

10. The apparatus of claim 1 wherein the electrolyte gel covers the at least one electrode element sensing portion.

11. The apparatus of claim 10 wherein the electrolyte gel covers substantially the entire backing element second surface.

12. The apparatus of claim 1 wherein the backing element is made of foam.

13. The apparatus of claim 1 wherein the backing element is made of vinyl.

14. The apparatus of claim 1 wherein the backing element is made of tape.

15. The apparatus of claim 3 wherein the electrode element includes a first sensing portion extending away from the aperture in a first direction generally parallel to the backing element second surface and a second sensing portion extending away from the aperture in a second direction generally parallel to the backing element second surface.

16. The apparatus of claim 15 wherein the electrolyte gel covers substantially the entire first and second electrode element sensing portion.

17. The apparatus of claim 16 wherein the portion of the backing element second surface that is not covered by electrolyte gel is covered by adhesive.

18. The apparatus of claim 1 where in the electrolyte gel is a liquid gel.

19. The apparatus of claim 18 further comprising an absorptive member coupled to the at least one electrode element sensing portion, sized and configured to cover substantially the entire electrode element sensing portions, a predetermined volume of liquid electrolyte gel being provided in the absorptive member;

20. The apparatus of claim 19 wherein the portion of the backing element second surface that is not covered by electrolyte gel is covered by adhesive.
